(19) United States
(12) Patent Application Publication
(10) Pub. No.: US 2007/0100264 A1
(43) Pub. Date: May 3, 2007

(54) Cold/heat treatment boot for ankles and lower legs

(76) Inventor: Linda Hanson, Whiting, IA (US)

Correspondence Address: Law Offices of Adam H. Jacobs, Suite 726, 1904 Farnam Street, Omaha, NE, 68102, US

(21) Appl. No.: 11/260,825

(22) Filed: Oct. 27, 2005

(51) Int. Cl. A61F 5/00

(52) U.S. Cl. 602/2

(57) ABSTRACT

A cold/heat treatment boot for ankles and lower legs includes a generally flexible lower leg-enclosing boot having an upwardly-extending leg section having left and right walls and a forward opening between the walls, and a generally flat sole plate mounted on and extending between bases of the walls. The forward opening releasably closeable via a closure device mounted on the boot to secure the boot on the leg and is further operative to permit the insertion of the foot and leg of a patient therein without requiring substantial flexion of the ankle of the patient and, further, at least two pockets are mounted on the boot, one on each of the walls of the leg section, to receive and support a heating or cooling device generally adjacent the lower leg and ankle of the patient whereby the patient can receive cold/heat treatment for their ankle and lower leg.

### BACKGROUND OF THE INVENTION

1. Technical Field

The present invention is directed to first-aid devices for ankles and legs and, more particularly, to a cold/heat treatment boot for ankles and lower legs which includes a generally flexible lower leg-enclosing boot having an upwardly extending leg section having left and right walls with a forward opening between the walls, a generally flat sole plate mounted on and extending between the left and right walls adjacent the bases thereof, at least two pockets, one mounted on each of the left and right walls of the leg section, the pockets operative to receive and support either a heating or cooling device generally adjacent the lower leg and ankle of the patient, and a closure device such as a clip or tie mounted on the boot generally adjacent the forward opening for releasably closing the forward opening with the lower leg and ankle of the patient therein, thus releasably securing the lower leg-encasing boot on the lower leg and ankle of the patient.

2. Description of the Prior Art

Injuries to the lower leg and ankles of individuals are quite common, particularly when the individual is involved in sports such as basketball, football, volleyball and baseball. Although very serious leg injuries such as broken bones and torn ligaments, tendons and muscles occasionally occur in athletes playing these sports, it is the relatively minor types of leg and ankle injuries which occur with far greater frequency. These include injuries such as muscle and ligament strains and sprains, from the twisted ankle to the strained calf muscle. However, it is most common to incur an injury to the ankle or lower leg area due to the stresses and strains placed on the ankle and lower leg during participation in the above-mentioned sports, as well as the stresses and strains encountered in many other activities. Regardless of the way in which the injury occurs, however, it is important to quickly apply treatment to the injury at the location of the injury, with the most common forms of treatment being application of cold and/or heat to the injury location to decrease swelling and promote healing of the injury.

Many devices are known for applying heat or cold to a particular injury location, including such devices as ice packs, heating packs, both electrical and chemical, and gel packs which may be either heated or cooled to apply the desired heating or cooling to the injury area. While these devices are designed to quickly and easily treat the afflicted location on the body, they unfortunately are not designed to be easily retained in that location adjacent the injured body part absent an additional securement device. There are several different types of securement devices which are proposed in the prior art for performing this function, each of which has inherent disadvantages. For example, the vast majority of physical therapists and trainers will secure the ice pack or heating pack to the afflicted area by use of an elastic bandage or the like which is wrapped around the ice pack or heating pad to secure the ice pack or heating pack in the desired location. These elastic bandages, however, are notoriously ineffective in securing the ice pack or heating pack as they are prone to slippage or accidental release of the secured ice pack or heating pack, thus requiring periodic adjustment and resetting of the elastic bandage on the afflicted body part. Furthermore, use of an elastic bandage in the ankle area is quite difficult due to the right angle of the ankle, and thus the elastic bandage is even less effective at securing the ice pack or heating pack to the afflicted location.

Other inventions are found in the prior art which attempt to address and solve the problem of securing an ice pack or heating pad to a particular body location, including those devices shown in Abare, U.S. Pat. No. 4,527,566, Lyles, U.S. Pat. No. 6,001,122, and Garcia, U.S. Pat. No. 5,873,903, but, as was mentioned previously, each of these prior art devices include inherent disadvantages and thus neither fully address nor solve the problem of securing an ice pack or heating pad to a body location, particularly adjacent or on the ankle of the individual. There is therefore a need for an improved cold/heat treatment boot for ankles and lower legs which addresses and solves many of the deficiencies found in the prior art.

Therefore, an object of the present invention is to provide an improved cold/heat treatment boot for ankles and lower legs.

Another object of the present invention is to provide a cold/heat treatment boot for ankles and lower legs which includes a generally flexible lower leg enclosing boot having an upwardly-extending leg section having left and right walls and a forward opening between the left and right walls, a generally flat sole plate mounted on and extending between the left and right walls adjacent the bases thereof, at least two pockets, one mounted on each of the left and right walls of the leg section and extending generally the entire length thereof to receive and support either a heating or cooling device such as an ice pack or heat pack, and a closure device mounted on the boot generally adjacent the forward opening thereof for releasably closing the forward opening with the lower leg and ankle of the patient therein to releasably secure the lower leg-enclosing boot on the lower leg and ankle of the patient.

Another object of the present invention is to provide a cold/heat treatment boot for ankles and lower legs which may be quickly and easily slipped onto the lower leg and ankle of the individual without requiring the individual to move, flex, or significantly adjust the position of the foot relative to the leg via the ankle in order to generally prevent additional injury to the injured ankle from unnecessary movement of the ankle post-injury.

Another object of the present invention is to provide a cold/heat treatment boot for ankles and lower legs in which the pockets in the left and right walls of the leg section are lined with a leak-proof lining material such as plastic to prevent leakage from the interior of the pocket, thus permitting the use of free ice in the cold/heat treatment boot of the present invention.

Another object of the present invention is to provide a cold/heat treatment boot for ankles and lower legs which is constructed of a fabric material for comfort and which is further designed to be disposable after use, thus rendering the cold/heat treatment boot of the present invention ideal for use in hospitals and on playing fields.

Finally, an object of the present invention is to provide a cold/heat treatment boot for ankles and lower legs which is relatively simple and inexpensive to manufacture and which is safe, efficient, and effective in use.

### SUMMARY OF THE INVENTION

The present invention provides a cold/heat treatment boot for ankles and lower legs which includes a generally flexible lower leg-enclosing boot having an upwardly-extending leg section having left and right walls and a forward opening between the left and right walls, and a generally flat sole plate mounted on and extending between the left and right walls adjacent the bases thereof. The forward opening between the left and right walls is further operative to permit the insertion of the foot and leg of a patient therein without requiring substantial flexion of the ankle of the patient and, further, at least two pockets are mounted on the boot, one mounted on each of the left and right walls of the leg section, the pockets operative to receive and support a heating or cooling device such as a heat pack or an ice pack generally adjacent the lower leg and ankle of the patient whereby the patient can receive cold/heat treatment for their ankle and lower leg. Finally, a closure device such as a clip or tie is mounted on the boot generally adjacent the forward opening for releasably closing the forward opening with the lower leg and ankle of the patient therein to releasably secure the lower leg-enclosing boot on the lower leg and ankle of the patient.

As thus described, the cold/heat treatment boot of the present invention provides many advantages over those devices found in the prior art. For example, because the boot may be placed on the lower leg and ankle of the individual merely by opening the forward opening and sliding the boot over the lower leg and ankle, the use of the present invention will not engender additional injury to the patient's lower leg and ankle which can be caused by unnecessary flexion and tension being applied or being required to be applied to the ankle of the individual while placing the treatment device thereon. Furthermore, because the present invention includes two pockets, one mounted on each of the left and right walls of the leg section, an injury taking place to either the inner or outer part of the lower leg and ankle may be treated separately without having to treat the entire lower leg, and thus focusing of the treatment may be easily accomplished. Also, because the present invention takes the form of a boot with a sole plate, the present invention may be worn by the patient even while the individual is walking on the injured leg, and thus the cold/heat treatment boot of the present invention functions somewhat as a walking cast, which is very useful in the treatment of many types of ankle and lower leg injuries. Finally, an important feature of the present invention is that it may be constructed of a fabric material which will support the heating or cooling pack adjacent the leg or ankle and then, after being used, the present invention may be discarded, thus rendering the cold/heat treatment boot disposable following a single use. This is invaluable for use in hospitals and the like, where regulations prohibit use of a treatment device with more than one patient absent expensive and extravagant sterilization procedures. It is thus seen that the cold/heat treatment boot of the present invention provides a substantial improvement over those devices found in the prior art.

### BRIEF DESCRIPTION OF THE DRAWINGS

FIG. 1 is a perspective view of the cold/heat treatment boot for ankles and lower legs of the present invention fitted onto the lower leg and ankle of a patient;

FIG. 2 is a perspective view of the cold/heat treatment boot for ankles and lower legs of the present invention;

FIG. 3 is a side elevational view of the present invention showing it fitted onto a patient's leg;

FIG. 4 is a detailed front elevational view of the present invention showing how cooling and heating devices fit into the pockets of the device;

FIG. 5 is a detailed front elevational view showing an alternative embodiment of the present invention having pocket linings to permit the use of bulk ice or the like; and

FIG. 6 is a perspective view of alternative embodiment of the cold/heat treatment boot for ankles and lower legs of the present invention which is designed to be disposable after a single use.

### DESCRIPTION OF THE PREFERRED EMBODIMENT

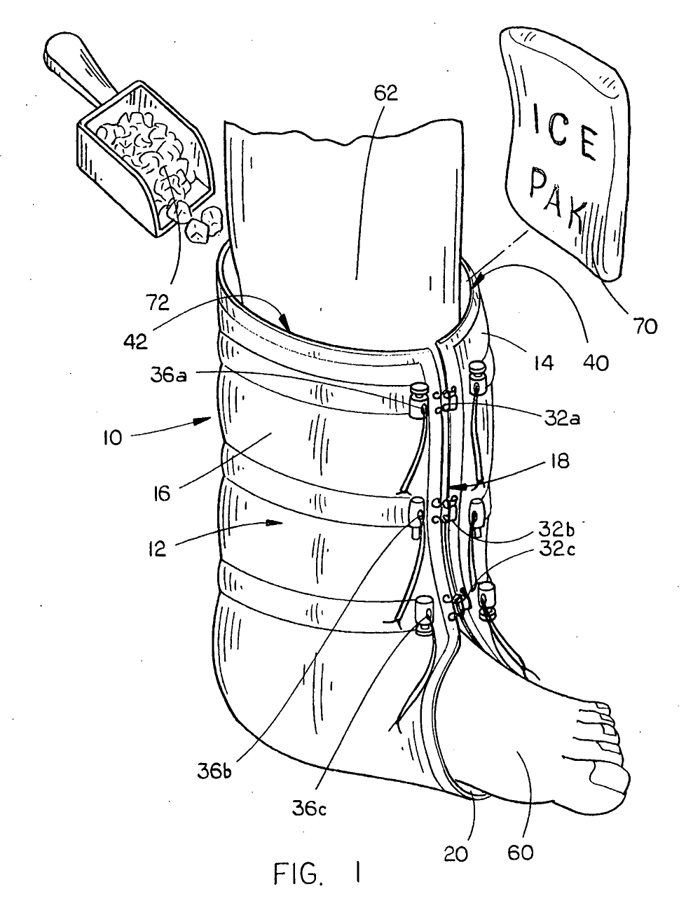

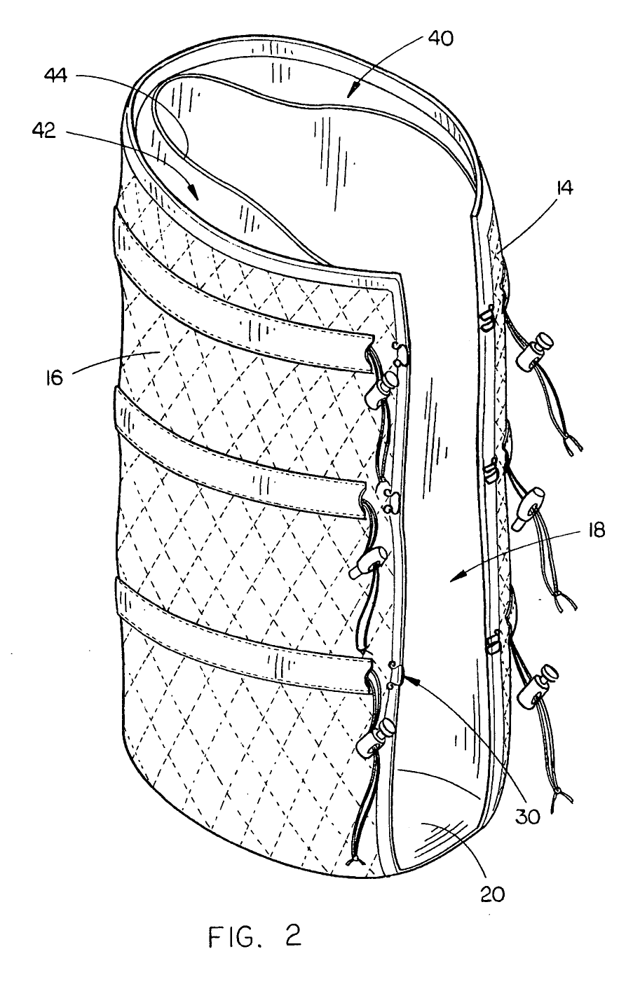

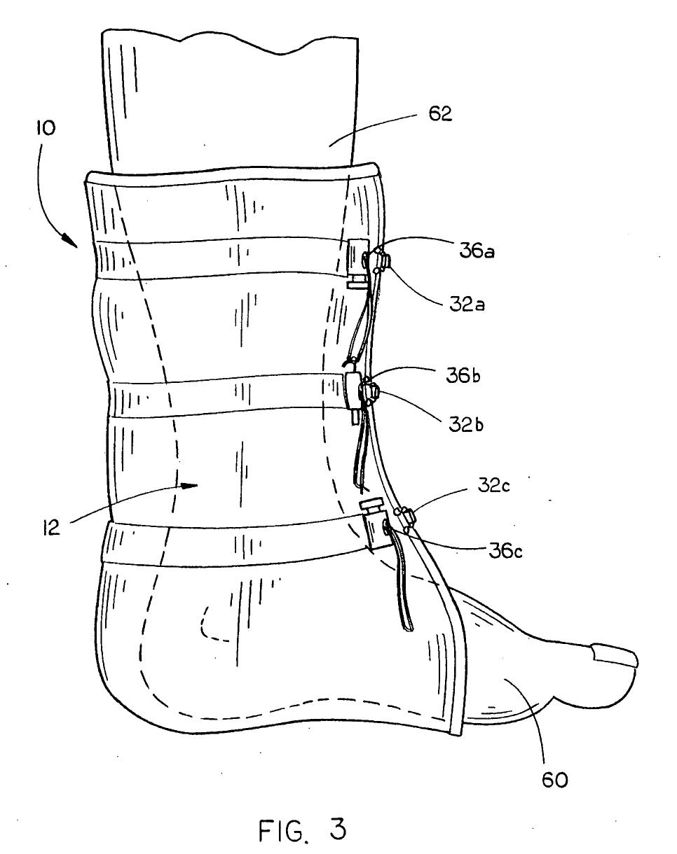

The cold/heat treatment boot 10 of the present invention is shown best in FIGS. 1-5 as including a generally flexible lower leg-enclosing boot 12 having left and right side walls 14 and 16 and a forward opening 18 positioned between the left and right side walls 14 and 16. In the preferred embodiment, the left and right side walls 14 and 16 would be joined to one another along the rear edges thereof to form the generally cylindrical shape of the lower leg-enclosing boot 12, as shown best in FIGS. 1 and 2, and further, it is preferred that left and right side walls 14 and 16 be constructed of a flexible fabric material which may be quilted or padded to increase the comfort level of the fabric material used in the lower leg-enclosing boot 12. In fact, it is expected that a cotton-based fabric will likely be used for the construction material of the left and right side walls 14 and 16, with the fabric material being treated to increase its resistance to staining and also render the fabric material generally waterproof, although such a treatment is not necessary to ensure proper functioning of the present invention. It is further preferred that the overall height of the lower leg-enclosing boot 12 be approximately twelve to twenty-four inches, depending on the size of the person with which the cold/heat treatment boot 10 of the present invention is to be used, with the diameter of the lower leg-enclosing boot 12 also varying with the size of the leg of the person to which the present invention is to be applied. Therefore, the overall size of the rectangular panels forming the left and right side walls 14 and 16 will vary to vary the overall size of the lower leg-enclosing boot 12.

Extending across and connecting the bases of the left and right side walls 14 and 16 is a generally flat sole plate 20, which, in the preferred embodiment, would be constructed of materials similar to that used in connection with left and right side walls 14 and 16 and would form the base or sole of the lower leg-enclosing boot 12, as shown best in FIGS. 1 and 2. The sole plate 20 may further include rubberized ridges 22 or the like formed on the underside of sole plate 20 to increase the frictional contact between the sole plate 20 and the floor surface on which the individual wearing the cold/heat treatment boot 10 of the present invention is walking. However, inclusion of such friction-increasing structures and substances on the underside of sole plate 20 is not critical to the present invention, although it has been found that their inclusion does decrease the likelihood of slippage of the sole plate 20 during walking.

Once the left side wall 14, right side wall 16, and sole plate 20 are connected to one another in the configuration shown in FIGS. 1 and 2, the main structural elements of the lower leg-enclosing boot 12 are completed. The lower leg-enclosing boot 12 may thus fit onto and generally enclose the foot 60 and lower leg 62 of the person wearing the cold/heat treatment boot 10 of the present invention, as shown best in FIG. 1. The lower leg-enclosing boot 12 would then be secured by closing the forward opening 18 via one or more closure devices 30, which, in the preferred embodiment, would include a plurality of releasable clasps 32a, 32b, and 32c having interlocking clasp sections each mounted on opposite edges of the left and right side walls 14 and 16 adjacent the forward opening 18, the releasable clasps 32a-c operative to close the forward opening 18 to releasably secure the lower leg-enclosing boot 12 on the foot 60 and lower leg 62 of the person wearing the cold/heat treatment boot 10 of the present invention. Of course, other types of closure devices 30 may be used in connection with the present invention, such as string ties, snaps, or any other type of closure device which will releasably secure the left and right side walls 14 and 16 to one another to close the forward opening 18, and such substitution of closure devices would be well known in the prior art.

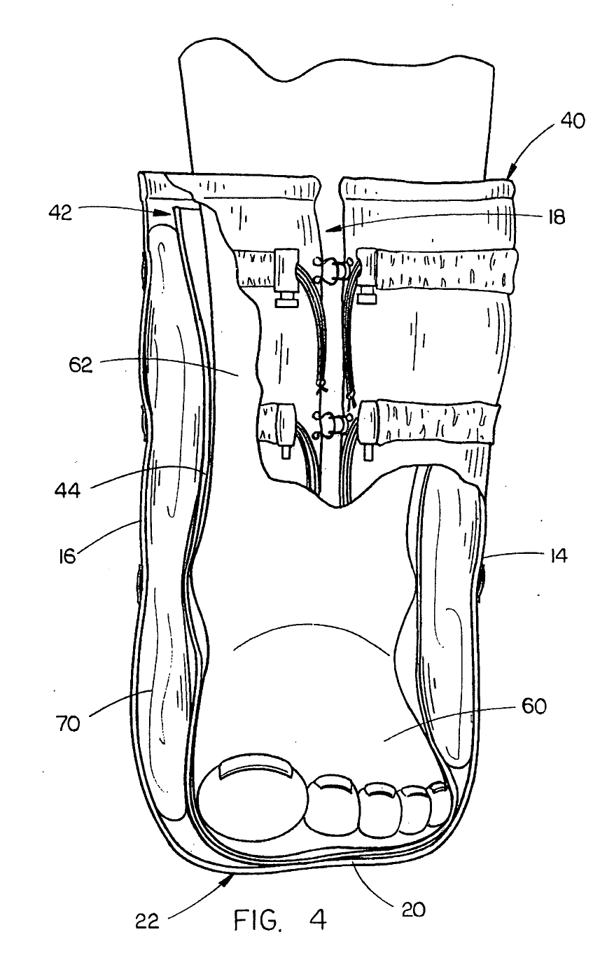

The truly inventive aspect of the present invention, however, has not yet been described, and specifically constitutes a pair of pockets, namely, a left side wall pocket 40 and right side wall pocket 42 which are mounted on the inner faces of left and right side walls 14 and 16, as shown best in FIGS. 2, 4, and 5. In the preferred embodiment, each of the left and right side wall pockets 40 and 42 would be formed in the similar manner, and therefore the description of right side wall pocket 42 should be understood to apply equally to left side wall pocket 40. Right side wall pocket 42 would include an inner wall 44 and an outer wall which is formed by right side wall 16, as shown best in FIG. 4. The inner wall 44 of right side wall pocket 42 would preferably be formed from a panel of generally flexible fabric material which is sufficiently thermally conductive to transfer the heat or cold from the heat or cold-producing device to be housed within the right side wall pocket 42. It has been found that a thin cotton-based fleece material provides a protective barrier for the person's foot 60 or lower leg 62 to prevent harmful contact with the heat or cold-producing device, yet will transfer a sufficient amount of the heat or cold therethrough to fulfill the injury treatment function of the present invention. However, the specific construction material used in connection with the inner wall 44 of right side wall pocket 42 is not particularly critical to the present invention so long as it provides a reasonable degree of comfort and protection for the foot 60 and lower leg 62 of the person while simultaneously permitting thermal transfer therethrough.

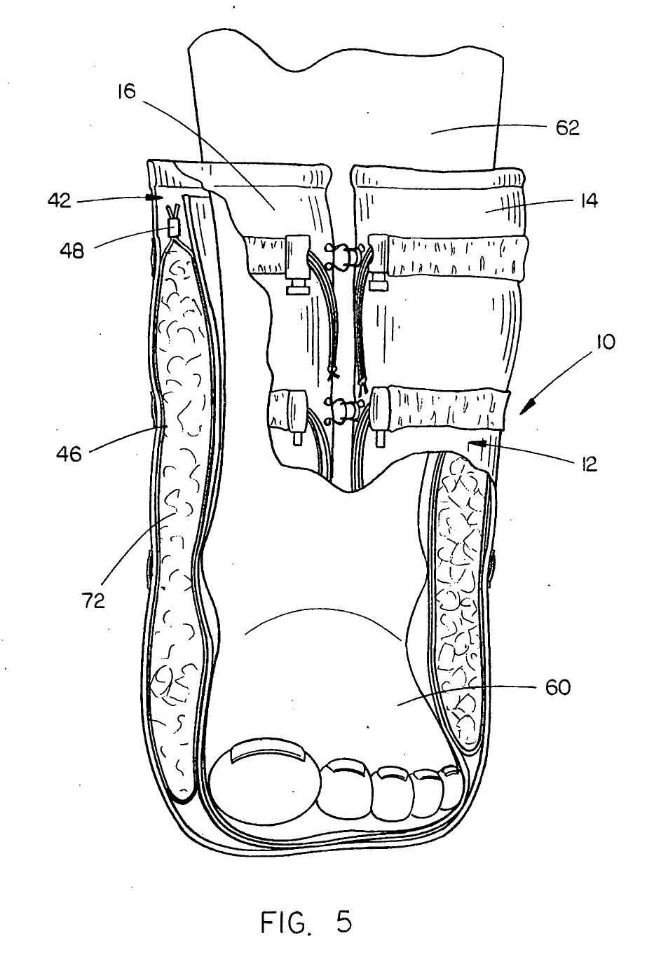

It may also be beneficial to line the inside of the right side wall pocket 42 with a waterproof lining material such as plastic or the like, which will conduct heat or cold therethrough, yet will prevent leakage of liquids into the fabric material forming the inner wall 44 and right side wall 16 of right side wall pocket 42. This plastic lining material 46 is shown best in FIG. 5 and may take the form of a sealable plastic bag which includes a zipper closure device 48 mounted at the top thereof such that when a heat or cold-producing material is placed within the right side wall pocket 42 within plastic lining 46, closure of the sipper closure device 48 will retain the material in the plastic lining 46 and prevent accidental release of the contents of the right side wall pocket 42 into the lower leg-enclosing boot 12.

It is expected that the heat or cold-producing devices used in connection with the cold/heat treatment boot 10 of the present invention will likely be in the form of ice packs 70, bags of ice 72, or, for heat, in the form of hot water bottles, chemical heat packs or other such cold or heat-producing device, any of which may be used with the cold/heat treatment boot 10 of the present invention so long as they fit within the left and right side wall pockets 40 and 42. For example, were it was found to be advantageous to apply cold to an injured portion of the lower leg 62 or foot 60, an ice pack 70 would be inserted down into the left side wall pocket 40 or right side wall pocket 42, as shown in FIG. 4, and because the left and right side wall pockets 40 and 42 extend substantially the entire height of the left and right side walls 14 and 16, no matter where the injury is on the lower leg 42 or foot 60 adjacent the ankle of the foot, the injury may be treated by application of cold thereto.

Alternatively, should the individual wish to use ice 72 instead of the ice packs 70, the ice 72 would be dropped into the left or right side wall pocket 40 and 42 which includes the plastic lining 46, as shown in FIG. 5. The plastic lining 46 would then be sealed via zipper closure device 48 and the ice 72 would thus be held within the left and right side wall pockets 40 and 42 to provide cold treatment to the afflicted portion of the foot 60 or lower leg 62 held within the lower leg-enclosing boot 12 of the present invention. In a similar manner, a chemical heat pack or other such heat-producing device could be inserted into either of the left and right side wall pockets 40 and 42 for application of heat to the afflicted part of the foot 60 or lower leg 62, and so long as the heat-producing device will fit within the left and right side wall pockets 40 and 42, it is usable with the cold/heat treatment boot 10 of the present invention.

Once the cold or heat-producing material is dropped into the left or right side wall pocket 40 and 42, and after the lower leg-enclosing boot 12 is slid onto the foot 60 and lower leg 62 of the patient via forward opening 18, the closure devices 30 would be closed and then one or more of the releasable tightening straps 36a, 36b, and 36c would be tightened to draw the left and right side walls 14 and 16 of the lower leg-enclosing boot 12 inwards to bring the inner wall 44 of the left and right side wall pockets 40 and 42 into contact with the foot 60 and lower leg 62 of the individual wearing the present invention. This ensures that the cold or heat being applied via the cold/heat treatment boot 10 of the present invention is being applied directly to the foot 60 or lower leg 62 of the individual, and further acts to secure the cold/heat treatment boot 10 of the present invention on the foot 60 and lower leg 62 of the patient. The advantage of having multiple releasable tightening straps 36a-c should be obvious in that wherever the heat or cold-producing device is located within the left and right side wall pockets 40 and 42, the appropriate releasable tightening straps 36a-c may be loosened or tightened to bring the inner wall 44 into contact with the foot 60 or lower leg 62 at that particular location, and thus the treatment options available through use of the cold/heat treatment boot 10 of the present invention are significantly expanded.

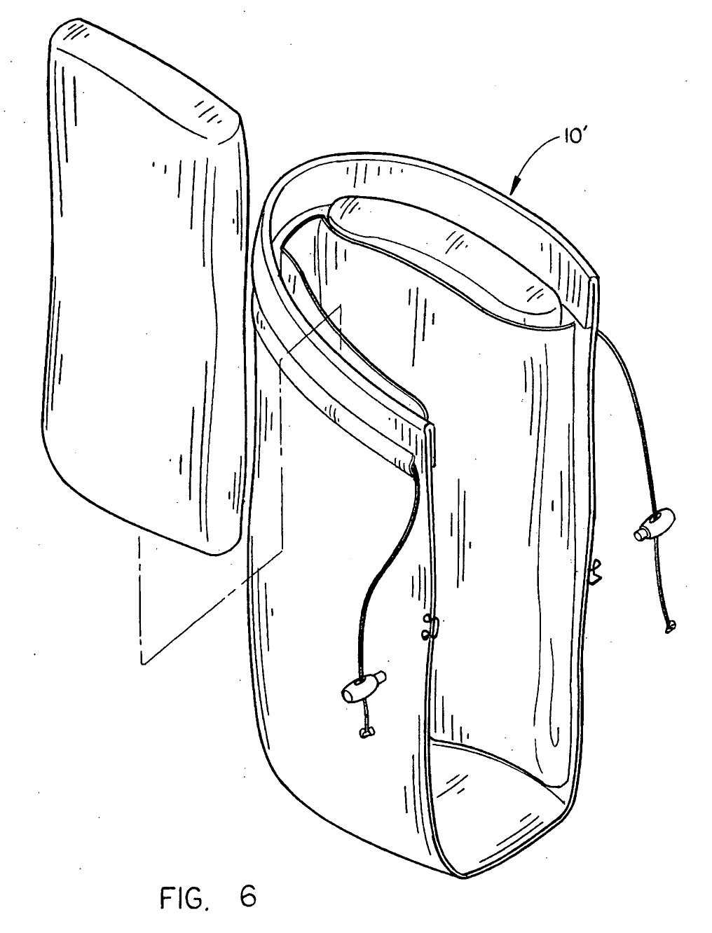

An alternative embodiment of the cold/heat treatment boot 10′ of the present invention is shown best in FIG. 6 as including substantially all of the same construction elements, with the significant difference being that the embodiment of FIG. 6 is specifically designed to be disposable after use. It is therefore preferred that the cold/heat treatment boot 10′ be constructed of an inexpensive fabric material, or may even be constructed of a high-strength paper material, with the use of the cold/heat treatment boot 10′ being substantially identical as that described in connection with the embodiments of FIGS. 1-5. The primary reason for disclosure of the embodiment of FIG. 6 is that, while many of the prior art devices disclose ways to secure heat and cold treatments to an afflicted portion of a foot or leg, they generally do not take into account the fact that hospitals, paramedics and other treating medical personnel cannot reuse a particular treatment device without washing and/or sterilizing the device between each and every use. This is particularly true when bodily fluids such as blood are released by the individual wearing the device to stain or otherwise be retained on the treatment device, and it is particularly likely that the cold/heat treatment boot 10 of the present invention will encounter release of bodily fluids due to its intended method of use. It is therefore very important that the cold/heat treatment boot 10′ be capable of being quickly and easily discarded after a single use in order to eliminate the need for cleaning and/or sterilization of the treatment boot each and every time it is used. It is believed that this is an important feature of the cold/heat treatment boot 10′ of the present invention, and therefore merits inclusion and eventual protection.

It is to be understood that numerous additions, modifications and substitutions may be made to the cold/heat treatment boot 10 of the present invention which fall within the intended broad scope of the appended claims. For example, the size, shape, and construction materials used in connection with the cold/heat treatment boot 10 are not particularly critical to the present invention so long as the intended functional features of the device are maintained. Furthermore, it should be noted that the size and shape of the left and right side wall pockets 40 and 42 may be modified or changed, although it is intended that the pockets 40 and 42 extend substantially the entire height of the left and right side walls 14 and 16 and substantially the width thereof also in order to permit the use of heat and cold-producing devices at virtually any position within the cold/heat treatment boot 10 in order to effectively treat injuries to the foot 60 adjacent the ankle and lower leg 62 of the patient. Finally, although the lower leg-enclosing boot 12 of the present invention has been described as including left and right side walls 14 and 16 and sole plate 20, it is perfectly acceptable to construct the entire lower leg-enclosing boot 12 from one or more separate pieces of construction material merely by bending and folding the construction material to form the approximate shape of the lower leg-enclosing boot 12, as shown best in FIGS. 1 and 2, and the use of such different sizes and shapes of construction panels for each of the left and right side walls 14 and 16 and sole plate 20 should be understood to constitute part of this disclosure and part of this invention.

There has therefore been shown and described a cold/heat treatment boot 10 which accomplishes at least all of its intended objectives.

I claim:

1. A cold/heat treatment boot for ankles and lower legs comprising:
a generally flexible lower leg-enclosing boot including;
an upwardly-extending leg section having left and right walls and a forward opening between said left and right walls;
a generally flat sole plate mounted on and extending between said left and right walls adjacent the bases thereof;
said forward opening operative to permit the insertion of the foot and leg of a patient therein without requiring substantial flexion of the ankle of the patient;
at least two pockets, one mounted on each of said left and right walls of said leg section, said at least two pockets operative to receive and support alternatively one of a heating and cooling device generally adjacent the lower leg and ankle of the patient whereby the patient can receive cold/heat treatment for their ankle and lower leg; and
closure means mounted on said boot generally adjacent said forward opening for releasably closing said forward opening with the lower leg and ankle of the patient therein to releasably secure said lower leg-enclosing boot on the lower leg and ankle of the patient.

2. The cold/heat treatment boot of claim 1 wherein said left and right walls generally conform to the shape of the leg on which said generally flexible lower leg-enclosing boot is secured.

3. The cold/heat treatment boot of claim 1 further comprising rubberized ridges formed on the underside of said generally flat sole plate operative to increase the frictional contact between said sole plate and the floor surface on which the individual wearing said cold/heat treatment boot is walking.

4. The cold/heat treatment boot of claim 1 wherein said at least two pockets each extend substantially the entire height of each of said left and right walls.

5. The cold/heat treatment boot of claim 1 further comprising waterproof lining sheet material mounted within said at least two pockets generally adjacent the walls thereof, said waterproof lining sheet material being thermally conductive yet operative to generally prevent leakage of liquids from said at least two pockets.

6. The cold/heat treatment boot of claim 1 wherein said cold/heat treatment boot is disposable after a single use.

7. The cold/heat treatment boot of claim 1 wherein said closure means comprises at least one releasable clasp having interlocking clasp sections each mounted on one of the left and right walls adjacent said forward opening, said at least one releasable clasp operative to releasably close said forward opening upon said interlocking clasp sections being interlocked.

8. The cold/heat treatment boot of claim 1 further comprising at least two releasable tightening straps extending circumferentially around said generally flexible lower leg-enclosing boot on said left and right walls, said at least two releasable tightening straps operative to be tightened to draw said left and right side walls of said lower leg-enclosing boot inwards whereby said at least two pockets are brought into contact with the foot and lower leg of the individual wearing said generally flexible lower leg-enclosing boot thereby ensuring that the cold/heat being applied via said cold/heat treatment boot is applied directly to the foot or lower leg of the individual.

9. The cold/heat treatment boot of claim 1 wherein said cold/heat treatment boot is constructed of a fabric material.

10. A cold/heat treatment boot for ankles and lower legs comprising:
a generally flexible lower leg-enclosing boot including;
an upwardly-extending leg section having left and right walls and a forward opening between said left and right walls;
a generally flat sole plate mounted on and extending between said left and right walls adjacent the bases thereof;
said forward opening operative to permit the insertion of the foot and leg of a patient therein without requiring substantial flexion of the ankle of the patient;
left and right pockets mounted on the inner face of each of said left and right walls of said leg section, said left and right pockets operative to receive and support alternatively one of a heating and cooling device generally adjacent the lower leg and ankle of the patient whereby the patient can receive cold/heat treatment for their ankle and lower leg;
said left and right pockets each extending substantially the entire height of each of said left and right walls; and
closure means mounted on said boot generally adjacent said forward opening for releasably closing said forward opening with the lower leg and ankle of the patient therein to releasably secure said lower leg-enclosing boot on the lower leg and ankle of the patient.

11. The cold/heat treatment boot of claim 10 further comprising waterproof lining sheet material mounted within said at least two pockets generally adjacent the walls thereof, said waterproof lining sheet material being thermally conductive yet operative to generally prevent leakage of liquids from said at least two pockets.

12. The cold/heat treatment boot of claim 10 wherein said cold/heat treatment boot is disposable after a single use.

13. A cold/heat treatment boot for ankles and lower legs comprising:
a generally flexible lower leg-enclosing boot including;
an upwardly-extending leg section having left and right walls and a forward opening between said left and right walls;
a generally flat sole plate mounted on and extending between said left and right walls adjacent the bases thereof;
said forward opening operative to permit the insertion of the foot and leg of a patient therein without requiring substantial flexion of the ankle of the patient;
at least two pockets, one mounted on each of said left and right walls of said leg section, said at least two pockets operative to receive and support alternatively one of a heating and cooling device generally adjacent the lower leg and ankle of the patient whereby the patient can receive cold/heat treatment for their ankle and lower leg;
closure means mounted on said boot generally adjacent said forward opening for releasably closing said forward opening with the lower leg and ankle of the patient therein to releasably secure said lower leg-enclosing boot on the lower leg and ankle of the patient; and
at least two releasable tightening straps extending circumferentially around said generally flexible lower leg-enclosing boot on said left and right walls, said at least two releasable tightening straps operative to be tightened to draw said left and right side walls of said lower leg-enclosing boot inwards whereby said at least two pockets are brought into contact with the foot and lower leg of the individual wearing said generally flexible lower leg-enclosing boot thereby ensuring that the cold/heat being applied via said cold/heat treatment boot is applied directly to the foot or lower leg of the individual.

14. The cold/heat treatment boot of claim 13 wherein said left and right walls generally conform to the shape of the leg on which said generally flexible lower leg-enclosing boot is secured.

15. The cold/heat treatment boot of claim 13 further comprising rubberized ridges formed on the underside of said generally flat sole plate operative to increase the frictional contact between said sole plate and the floor surface on which the individual wearing said cold/heat treatment boot is walking.

16. The cold/heat treatment boot of claim 13 wherein said at least two pockets each extend substantially the entire height of each of said left and right walls.

17. The cold/heat treatment boot of claim 13 further comprising waterproof lining sheet material mounted within said at least two pockets generally adjacent the walls thereof, said waterproof lining sheet material being thermally conductive yet operative to generally prevent leakage of liquids from said at least two pockets.

18. The cold/heat treatment boot of claim 13 wherein said cold/heat treatment boot is disposable after a single use.
